(19) United States
(12) Patent Application Publication
(10) Pub. No.: US 2006/0159727 A1
(43) Pub. Date: Jul. 20, 2006

(54) Prevention of Systemic Microbial Infection

(76) Inventor: Julio Lionel Pimentel, Buford, GA (US)

Correspondence Address: JULIO LIONEL PIMENTEL, 3206 WINDGATE DR., BUFORD, GA, 30519, US

(21) Appl. No.: 10/905,797

(22) Filed: Jan. 20, 2005

(51) Int. Cl. A23K 1/165; A61K 31/12; A61K 31/05

(52) U.S. Cl. 424/442; 514/690; 514/731

(57) ABSTRACT

Composition and methods for the prevention of systemic bacterial infection comprising the oral administration of food, feed and/or major food/feed ingredients or water treated with a terpene composition, water and/or surfactant is disclosed. The composition of the invention can be administered as a spray mist over food and feed while mixing, drop-wise as in a medicator (dosificator) in water or mix as a component of the diet. The composition of the invention prevents bacterial colonization of internal organs and eggs.

### FIELD OF THE INVENTION

A method to prevent systemic microbial infection in animals and humans by orally administering water, food, feed and/or major food/feed ingredient treated with a composition comprising citral, eugenol, I-carvone and surfactant or an emulsion comprising citral, eugenol, I-carvone, surfactant and water.

### DISCUSSION OF THE BACKGROUND

Foodborne illness is a common problem for millions of people around the world. Foodborne illness problems due to Campylobacter spp., Shigella spp., Listeria monocytogenes and Yersenia enterolitica have fallen between 1996 and 2002 in the United States; but Salmonella spp. and E. coli infections have remained constant. CDC statistics suggest that 76 million become sick each year due to consumption of undercooked meat, eggs, shellfish, unpasteurized dairy products and unwashed vegetables. Food producing animals are the major reservoir of non-typhi serotypes of Salmonella enterica, which causes an estimated 1.4 million illnesses, 16,400 hospitalizations and 580 deaths/year in the United States.

The incidence of Salmonella, E. coli and Enterococcus varies depending on the type of ingredient used on animal feed manufacturing. There is high incidence of Salmonella in rendered animal food (35%) than in plant based food (5%). The incidence of E. coli is similar in both animals and plant derived foods (40%), but the incidence of Enterococcus is 80% in animal and 91% in plant derived foods.

Salmonella is a facultative, intracellular pathogen capable of infesting humans and animals resulting in enteric fever, bacteremia and gastroenteritis. Salmonella is an invasive organism that can escape the confines of the intestine and transported by the blood to the internal organs. Following oral ingestion, salmonella penetrates the mucosal epithelium of the small intestine where macrophages begin the eradication process but when it fails, internalization and dissemination of salmonella occurs in other organs (Henderson, S. et. al., 1999, Early events in the pathogenesis of avian salmonellosis, Infec. Immun. 67(7): 3580-3586).

The majority of the salmonellosis present in humans has been traced to the consumption of eggs. Two days after hens were orally challenged, salmonella was detected in spleen, liver, heart, gall bladder tissues, intestinal tissues and various sections of the oviduct. The principal site of infection in eggs appears to be the upper oviduct. In the eggs contents the most important site of contamination are either the outside of the vitelline membrane or the albumen surrounding it. In eggs stored at 20° C. yolk invasion is uncommon until eggs have been stored for 3 weeks; at 30° C. growth is faster and in fewer days (Humphrey, T. J. et. al, 1994, Contamination of egg shell and contents with Salmonella enteritidis, Int. J. Food Microbiol 21 (1-2): 31-40). Factors present in eggs helped maintain lower salmonella level in freshly laid eggs content (0.6% incidence) even though eggs from the oviduct of the same hen showed higher salmonella levels (29% incidence); these factors may include antibodies, antibacterial enzymes and iron-sequestering and bacterial protease-inhibiting proteins in yolk and albumen (Keller, L. H. et. al., 1995, Salmonella enteritidis colonization of the reproductive tract and forming and freshly laid eggs of chickens. Infec. Immun. 63(7): 2443-2449).

Another problem in the egg industry is the stress caused by feed withdrawal to induced molting. This procedure causes and increases susceptibility to salmonella infection marked by an increased intestinal shedding and dissemination of salmonella to internal organs such as liver, spleen and ovary. When feed is withdrawn Lactobacilli that are the predominant colonizers of the crop diminish in number and the concentration of lactate and volatile fatty acids decreases therefore affecting crop pH and increasing pathogen colonization (Durant, J. A., et. al., 1999, Feed deprivation affects crop environment and modulates Salmonella enteritidis colonization and invasion of leghorn hens. Appl. Environ. Microbiol 65: 1919-1923)

The incidence of salmonella contamination in animal feed is higher mash form than in pellet form. Pelletization is done at high temperature and high-pressure conditions, this process reduces the number not only of salmonella but also other bacteria. Other treatments are the use of organic acids, formaldehyde and irradiation. The elimination of salmonella with organic acids requires high levels of treatments for a cost that for the animal industry is difficult to bare. In the case of formaldehyde, the possible link of this chemical with the incidence of cancer has brought a negative feeling on its use. The use of irradiation of feed is not cost effective and non-consumer friendly.

Various methods and programs have been pursued to reduce Salmonella enteriditis (Se) infection, including the use of the Hazard Analysis Critical Control Point (HACCP) system, in both pre-harvest and post-harvest settings but all of them have their limitations.

The efficacy of vaccination against salmonella in hens is limited to homologous or antigenically related serovars as salmonella serovar differ significantly in their flagellar and somatic antigens.

U.S. Pat. No. 6,645,515 describes a bacteriostatic composition for salmonella comprising a fermented broth obtained from the fermentation of lactic acid bacteria.

U.S. Pat. No. 6,214,335 describes a method to reduce enteropathogenic bacteria by feeding a bacteria cultured from intestinal scrapings of healthy birds and preventing colonization by enteropathogenic bacteria including Salmonella, E. coli and Campylobacter.

U.S. Pat. No. 6,656,463 provides a method to reduce or salmonella dissemination in swine by feeding a phage prior of harvesting the animals.

The use of two phenolic, one quaternary ammonium, one quaternary ammonium with formaldehyde and one hypochlorite based products were effective on decreasing salmonella in in-vitro test (Davison, S. et. al, 2003, The role of disinfectant resistant of Salmonella enterica serotype enteritidis in recurring infection in Pennsylvania egg quality assurance program monitored flocks, Avian Diseases 47(1) 143-148).

Methanol extract of herbs helped to increase survival and specific growth rate of shrimp and reduced bacteria load i.e. Salmonella typhi, Pseudomona aeruginosa, Staphylococcus aureus and Vibrio spp. (Citarasu, T. et. al., 2003, Influence of the antibacterial herbs, Solanum trilobatum, Andrographis paniculata and Psoralea corylifolia on the survival, growth and bacterial load of Penaeus monodon post larvae. Aquaculture Int. 11(6): 581-595).

Sodium percabonate is a powerful oxidizer that is used as antimicrobial in feed at a level of 1-2% of the diet.

Salmonella-challenged pigs when administered chlorate ions through water before slaughter had reduced bacteria counts in the intestine contents and lymph tissue (Anderson, R. C. et. al. 2004, Effect of drinking-water administration of experimental chlorate ion preparations on Salmonella enterica serovar Typhimurium colonization in weaned and finished pigs, Vet. Res. Comm. 28(3): 179-189).

Chlorate treatment is recommended for E. coli and Salmonella since these bacteria has the enzyme nitrate reductase that reduces chlorate to chlorite, which has antimicrobial properties.

The only option when the infection is systemic (blood and organs) is the use of antibiotics, none of the products now available in the market have the capacity of been absorbed through the intestine and be carried through the blood to all infected areas. The use of antibiotics as treatment of infection have given good results but with the problem with antibiotic resistance and residues in meat and eggs, its use have been restricted only for the treatment of infections and not as growth promoter. The European Community has banned the use of 5 antibiotics and in the Unites States the FDA is banning the use of fluoroquinolone in animals due to the development of Campylobacter-resistant to this antibiotic. Therefore new user-friendly alternatives are always been sought.

Terpenes are Generally Recognized as Safe (GRAS) and they are widespread in nature, mainly in plants as constituents of essential oils. Examples of terpenes include citral, pinene, nerol, b-ionone, geraniol, carvacrol, eugenol, I-carvone, terpeniol, anethole, camphor, menthol, limonene, nerolidol, farnesol, phytol, carotene, squalene, thymol, tocotrienol, perillyl alcohol, borneol, myrcene, simene, carene, terpenene, linalool and others.

Terpenes have been found to inhibit the in vitro growth of bacteria and fungi (Chaumont J. P. and D. Leger, 1992, Campaign Against Allergic Moulds in Dwellings, Inhibitor Properties of Essential Oil Geranium “Bourbon,” Citronellol, Geraniol and Citral, Ann. Pharm. Fr 50(3): 156-166), and some internal and external parasites (Hooser, S. B., V. R. Beasly and J. J. Everitt, 1986, Effects of an Insecticidal Dip Containing D-limonene in the Cat, J. Am. Vet. Med. Assoc. 189(8): 905-908). Geraniol was found to inhibit growth of Candida albicans and Saccharomyces cerevisiae strains by enhancing the rate of potassium leakage and disrupting membrane fluidity (Bard, M., M. R. Albert, N. Gupta, C. J. Guuynn and W. Stillwell, 1988, Geraniol Interferes with Membrane Functions in Strains of Candida and Saccharomyces, Lipids 23(6): 534-538). B-ionone has antifungal activity which was determined by inhibition of spore germination and growth inhibition in agar (Mikhlin E. D., V. P. Radina, A. A. Dmitrossky, L. P. Blinkova, and L. G. Button, 1983, Antifungal and Antimicrobial Activity of Some Derivatives of Beta-Ionone and Vitamin A, Prikl Biokhim Mikrobiol, 19: 795-803; Salt, S. D., S. Tuzun and J. Kuc, 1986, Effects of B-ionone and Abscisic Acid on the Growth of Tobacco and Resistance to Blue Mold, Mimicry the Effects of Stem Infection by Peronospora Tabacina, Adam Physiol. Molec. Plant Path 28:287-297). Teprenone (geranylgeranylacetone) has an antibacterial effect on H. pylori (Ishii, E., 1993, Antibacterial Activity of Terprenone, a Non Water-Soluble Antiulcer Agent, Against Helicobacter Pylori, Int. J. Med. Microbiol. Virol. Parasitol. Infect. Dis. 280(1-2): 239-243). Solutions of 11 different terpenes were effective in inhibiting the growth of pathogenic bacteria in in vitro tests (Kim, J., M. Marshall and C. Wei, 1995, Antibacterial Activity of Some Essential Oil Components Against Five Foodborne Pathogens, J. Agric. Food Chem. 43: 2839-2845). Diterpenes, i.e., trichorabdal A (from R. Trichocarpa), have shown a very strong antibacterial effect against H. pylori (Kadota, S., P. Basnet, E. Ishii, T. Tamura and T. Namba, 1997, Antibacterial Activity of Trichorabdal A from Rabdosia Trichocarpa Against Helicobacter Pylori, Zentralbl. Bakteriol 287(1): 63-67). Owawunmi, G. O., 1989 (Evaluation of the Antimicrobial Activity of Citral, Letters in Applied Microbiology 9(3): 105-108), showed that growth media with more than 0.01% citral reduced the concentration of E. coli, and at 0.08% there was a bactericidal effect. U.S. Pat. No. 5,673,468, teaches a terpene formulation, based on pine oil, used as a disinfectant or antiseptic cleaner. U.S. Pat. No. 5,849,956, teaches that a terpene found in rice has antifungal activity. U.S. Pat. No. 5,939,050, teaches an oral hygiene antimicrobial product with a combination of 2 or 3 terpenes that showed a synergistic effect. Several U.S. patents (U.S. Pat. Nos. 5,547,677, 5,549,901, 5,618,840, 5,629,021, 5,662,957, 5,700,679, 5,730,989) teach that certain types of oil-in-water emulsions have antimicrobial, adjuvant, and delivery properties. U.S. Pat. No. 5,906,825 teach us of antimicrobial agents from plant and herbal extracts for use on food-contact surfaces. U.S. Pat. No. 5,591,467 teach us of a formaldehyde-based antimicrobial feed additive which has d-limonene to mask the smell of formaldehyde. All these references and patents do not suggest the use of terpene mixtures or terpene suspensions for the prevention of systemic bacterial infection in humans and animals.

### SUMMARY OF THE INVENTION

In accordance with the purpose(s) of this invention, as embodied and broadly described herein, this invention relates to a water, feed and major feed ingredients additive for the prevention of systemic bacterial infection in animals.

The invention is related to the field of anti-infectives. The present invention provides compositions and methods for the prevention of systemic bacterial infection that avoids some drawbacks found in previous methods.

The present invention provides a composition for the prevention of systemic bacterial infection comprising an effective amount of effective terpenes. The composition can be a solution. The composition can be a mixture. The composition can be an emulsion. The composition can further comprise a carrier, e.g., water. The composition can further comprise a surfactant.

The composition may be a solution of terpene and surfactant. The composition may be an oil/water emulsion of terpene, surfactant and water. The terpenes of the composition comprise citral, eugenol and 1-carvone, or mixtures thereof.

The composition is effective against various infective agents including bacteria, viruses, mycoplasmas, and/or fungi present in drinking water, food, feed and major food/feed ingredients. The composition is further effective against Salmonella spp, Clostridia spp, Campylobacter spp, Listeria spp., Shigella spp., Yersenia spp. and E. coli in egg producing animals.

Additional advantages will be set forth in part in the description, which follows below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF THE PREFERRED EMBODIMENTS

Before the present compositions are disclosed and described, it is to be understood that this invention is not limited to specific methods. It is also to be understood that the terminology used herein is for the purpose of describing particular aspects only and is not intended to be limiting.

In this specification and in the claims, which follow, reference will be made to a number of terms, which shall be defined to have the following meanings

Definitions

A “volume percent” of a component, unless specifically stated to the contrary, is based on the total volume of the formulation or composition in which the component is included.

An “effective terpene” of the composition can comprise, for example, citral, pinene, nerol, b-ionone, geraniol, carvacrol, eugenol, I-carvone, terpeniol, anethole, camphor, menthol, limonene, nerolidol, farnesol, phytol, carotene, squalene, thymol, tocotrienol, perillyl alcohol, borneol, myrcene, simene, carene, terpenene, linalool, other biocidal terpenes or mixtures thereof. More specifically, the terpenes can comprise citral, I-carvone, eugenol, or mixtures thereof.

By the term “effective amount” of a compound is meant such amount capable of performing the function of the compound or property for which an effective amount is expressed, such as a non-toxic but sufficient amount of the compound to provide the desired function, i.e., anti-infective. Thus an appropriate effective amount may be determined by one of ordinary skill in the art using only routine experimentation

Several formulations can be obtained by utilizing biocidal terpenes without departing from the principle of the present inventions. Formulations can vary not only in the concentration and type of terpenes but also in the type of surfactant and water concentration used. This invention can be modified in several ways by adding new terpenes and surfactants.

The present invention includes methods of making the compositions and methods of using the compositions

Composition(s)

The compositions of the present invention comprise isoprenoids. More specifically, the compositions of the present invention comprise terpenoids. Even more specifically, the compositions of the present invention comprise terpenes found naturally in essential oils or synthetically elaborated.

The composition can comprise between about 10 ppm and about 10,000 ppm of the terpene, specifically 100, 250, 500, 1000 or 5000 ppm

A composition of the present invention comprises an effective amount of effective terpenes. Plant extracts or essential oils containing terpenes can be used in the compositions of this invention as well as the more purified terpenes. Terpenes are readily commercially available or can be produced by various methods known in the art, such as solvent extraction or steam extraction/distillation. Natural or synthetic terpenes are expected to be effective in the invention.

The surfactant can be non-ionic, cationic, or anionic. Examples of surfactant include polysorbate 20, polysorbate 80, polysorbate 40, polysorbate 60, polyglyceryl ester, polyglyceryl monooleate, decaglyceryl monocaprylate, propylene glycol dicaprilate, triglycerol monostearate, Tween™ 20, Span™ 20, Span™ 40, Span™ 60, Span™ 80, other natural/synthetic surfactant or mixtures thereof.

The composition can comprise 1 to 100% terpenes, 0 to 99% surfactant and 0 to 99% water. More specifically the composition can comprise about 0.01% to about 95% terpenes and about 1-15% surfactant

Specific compositions can include 10-1000 ppm terpenes in drinking water with 20% citral, 35% I-carvone, 40% eugenol, and 5% Tween™ 80. Concentrations of terpene from 10 to 20000 ppm can be used as effective concentrations in the compositions and methods of the current invention.

Specific compositions can include 10-1000 ppm terpenes in saline with 20% citral, 35% 1-I-carvone, 40% eugenol, and 5% Tween™ 80. Concentrations of terpene from 10 to 20000 ppm can be used as effective concentrations in the compositions and methods of the current invention.

Specific compositions can include 10-1000 ppm terpenes in distilled water with 20% citral, 35% I-carvone, 40% eugenol, and 5% Tween™ 80. Concentrations of terpene from 10 to 20000 ppm can be used as effective concentrations in the compositions and methods of the current invention.

Specific compositions can include 10-1000 ppm terpenes in edible liquids with 20% citral, 35% 1-carvone, 40% eugenol, and 5% Tween™ 80. Concentrations of terpene from 10 to 20000 ppm can be used as effective concentrations in the compositions and methods of the current invention.

Terpenes will decompose to CO₂ and water. This decomposition or break down of terpenes is an indication of the safety and environmental friendliness of the compositions and methods of the invention.

The terpenes, and surfactants of the invention may be readily purchased or synthesized using techniques generally known to synthetic chemists.

Methods

The invention includes a method of making a terpene-containing composition that is effective as an anti-infective.

The present invention is effective against any of these classifications of infective agents present in water, food, feed and major food/feed ingredients, in particular, bacteria, mycoplasmas, virus and fungi. Examples of these infective agents are Staphylococcus aureus, Aspergillius fumigatus, Mycoplasma iowae, Sclerotinta homeocarpa, Rhizoctonia solani, Colletotrichum graminicola, Penicillum spp., Mycoplasma pneumoniae, E. coli, Salmonella spp., Clostridia spp., Campylobacter spp. and others. The compositions and methods of the present invention are effective in preventing many, if not all, of these infections in a great variety of subjects, including humans, other mammals and avians.

The composition of this invention can be administered by a variety of means. For example, sprayed onto food/feed, sprayed onto water, applied to surfaces where water and feed are stored for future uses or consumed daily, added drop wise through a standard medicator or water dosificator, for example in starter, grower, finisher, breeding, and other animal houses.

Throughout this application, various publications and patents are referenced. The disclosures of these publications in their entireties are hereby incorporated by reference into this application in order to more fully describe the state of the art to which this invention pertains.

Methods for making specific and exemplary compositions of the present invention are described in detail in the Examples below.

### EXAMPLES

The following examples are put forth so as to provide those of ordinary skill in the art with a complete disclosure of how the compositions claimed herein are made and evaluated, and are intended to be purely exemplary of the invention and are not intended to limit the scope of what the inventor regard as his invention. There are numerous variations and combinations of the compositions and conditions for making or using them, e.g., component concentrations, and other ranges and conditions that can be used to optimize the results obtained from the described compositions.

The following terpene compositions were utilized in some of the examples now disclosed.

TABLE 1
Terpene Compositions, Formula Name and Percentage

| Ingredient | PL | B | FP | FP-20 |
|---|---|---|---|---|
| Citral | 20 | 70 | 30 | 30 |
| L-I-carvone | 35 | 10 | 30 | 30 |
| Eugenol | 40 | 0 | 30 | 30 |
| B-ionone | 0 | 10 | 0 | 0 |
| Tween 80 ™ | 5 | 10 | 10 | 0 |
| Span 20 ™ | 0 | 0 | 0 | 10 |

### Example 1

This example shows a comparison between several antimicrobials and four terpene compositions (table 1) on a susceptibility test. An agar plate was uniformly inoculated with salmonella and a paper disk impregnated with a fixed concentration of test material was placed on top of the agar surface. Growth of the organism and diffusion of the test material resulted in a circular zone of inhibition, the width of the zone is a function of the inhibitory response of the test material. The bacteria used was Salmonella enteriditis phage type VIII (SePT8). The study was conducted for 2 days. A disk soaked in sterile water was used as control.

TABLE 2
Zone of inhibition by different antimicrobials

| Product | Zone of Inhibition (mm) |
|---|---|
| Control | 0.000 |
| Probiotics | 0.278 |
| Yeast Extract | 0.778 |
| Calsporin | 1.000 |
| Copper Sulfate | 1.444 |
| Gentamycin | 1.300 |
| Baytril | 1.400 |
| Terpene PL | 1.000 |
| Terpene IB | 1.000 |
| Terpene FP | 1.000 |
| Terpene FP-20 | 1.000 |

### Example 2

This example shows the concentration of salmonella in selected organs when chicks were fed the test material described in example 1. Newly hatched chicks were fed a diet supplemented with the test materials, at 7 days of age, chicks were challenged 0.2 ml of a 2×10⁹ salmonella culture. Chicks were sacrificed at 14 days of age (1 week after bacterial challenge), their organs dissected and cultured to determine bacterial counts.

TABLE 3
Effect of antimicrobials on the concentration of salmonella in different organs (cfu)

| | Positive | Copper | Yeast | | | Competitive | | | Terpene | Terpene | Terpene |
|---|---|---|---|---|---|---|---|---|---|---|---|
| Tissue | Control | Sulfate | Extract | Calsporin | Probiotics | Exclusion | Baytril | Terpene B | FP | FP-20 | PL |
| Liver | 700 | 11 | 218 | 211 | 99 | 23 | 0 | 7 | 5 | 7 | 3 |
| Spleen | 1000 | 10 | 742 | 91 | 71 | 0 | 5 | 4 | 5 | 5 | 4 |
| Duodenum | 700 | 19 | 115 | 52 | 158 | 116 | 0 | 6 | 7 | 10 | 8 |
| Ceca | 1000 | 54 | 241 | 185 | 158 | 144 | 18 | 34 | 30 | 114 | 55 |
| Colon | 1000 | 74 | 473 | 192 | 229 | 454 | 0 | 16 | 24 | 75 | 44 |

### Example 3

This example shows the incidence of salmonella in livers of chicks fed terpenes and challenged with salmonella. Newly hatched chicks were fed diets supplemented with 250 ppm of terpene formulations as in example 1; at 7 days of age, each chick was challenged with 0.2 ml of a 2×10⁹ salmonella culture. Chicks were sacrificed at 14 days of age, the liver was dissected and tested for the presence of salmonella. The results were as follow:

TABLE 4
Incidence of Salmonella in Chick livers

| Treatment | Incidence |
|---|---|
| Negative Control | 0/12 |
| Positive Control | 12/12 |
| Terpene B | 0/12 |
| Terpene FP | 11/12 |
| Terpene FP20 | 0/12 |
| Terpene PL | 8/12 |

### Example 4

This example shows the incidence of salmonella in livers of pullets fed terpenes and challenged with salmonella. 17-week old pullets were fed diets supplemented with 250 ppm of terpene formulations as in example 1; seven days after, each pullet was challenged with 2 ml of a 2×10⁹ salmonella culture. Pullets were sacrificed at one week after challenge, the liver was dissected and tested for the presence of salmonella. The results were as follow:

TABLE 5
Incidence of Salmonella in Pullet livers

| Treatment | Incidence |
|---|---|
| Positive Control | 7/10 |
| Negative Control | 2/10 |
| Terpene B | 3/10 |
| Terpene PL | 3/10 |

It will be apparent for those skilled in the art that a number of modifications and variations may be made in the present invention without departing from the scope of the invention. It is intended that the specification and examples be considered as exemplary only, with a true scope and spirit of the invention being indicated by the following claims.

1. A method for reducing the incidence of systemic salmonella infections in animals by orally administering food, water, feed and/or major food/feed ingredients treated with a composition; said composition comprising terpenes, surfactant and water.

2. The method of claim 1, wherein the terpenes comprise citral, eugenol and 1-carvone; and the surfactant is polysorbate-80.

3. (canceled)

4. (canceled)

5. (canceled)

6. (canceled)

7. The method of claim 1, wherein the food, feed, major food/feed ingredients or water treated with said composition prevents colonization of salmonella in blood, liver, spleen and ovary.

8. (canceled)

9. The method of claim 1, wherein the food, feed, major food/feed ingredients or water treated with the composition prevents internal salmonella contamination of eggs.

10. The method of claim 1, wherein the food, feed and/or major food/feed ingredients or water treated with the composition prevents salmonella contamination of animal produce.

11. A method for reducing the incidence of digestive system salmonella infections in humans that consumed processed meat and eggs obtained from animals orally administered the composition described on claim 1.
